(19) United States
(12) Patent Application Publication
(10) Pub. No.: US 2024/0299219 A1
(43) Pub. Date: Sep. 12, 2024

(54) TAPED ABSORBENT ARTICLE WITH FRONT AND BACK ELASTIC WAISTBANDS

(71) Applicant: The Procter & Gamble Company, Cincinnati, OH (US)

(72) Inventors: Fengchun YE, Beijine (CN); Haoran ZHAO, Beijing (CN)

(21) Appl. No.: 18/663,254

(22) Filed: May 14, 2024

Related U.S. Application Data
(63) Continuation of application No. PCT/CN2021/141088, filed on Dec. 24, 2021.

(51) Int. Cl. A61F 13/494; A61F 13/15; A61F 13/49; A61F 13/53

(52) U.S. Cl. CPC A61F 13/49466; A61F 13/15707; A61F 13/49011; A61F 13/53

(57) ABSTRACT

A taped absorbent article having a front elastic waistband adjacent its front waist edge and a back elastic waistband adjacent the back waist edge. The front elastic waistband has a Front Waistband Elastic Force in the range from about 0.03N to about 0.32N, the back elastic waistband having a Back Waistband Elastic Force which is higher than the Front Waistband Elastic Force, as measured by Waist Band Elastic Force Measurement Method disclosed herein.

### CROSS-REFERENCE TO RELATED APPLICATIONS

This application is a continuation of and claims priority under 35 U.S.C. § 120 to PCT Patent Application Serial No. PCT/CN2021/141088 filed on Dec. 24, 2021, the entire disclosure of which is hereby incorporated by reference.

### FIELD

The invention relates to absorbent articles for personal hygiene, such as baby diapers and adult incontinence products.

### BACKGROUND

Disposable absorbent articles of the diaper type comprise an absorbent core disposed between a liquid permeable topsheet and a liquid impermeable backsheet. Urine is acquired into the absorbent article through the topsheet and is absorbed by the absorbent material. Taped diapers comprise a pair of back ears with tapes that can be releasably attached to a landing zone on the front of the diapers. The back ears are typically elasticized so that the tension around the waist can be adjusted by the caregiver. Pant diapers on the other hand have side seals and are put on and worn like underwear. While it is common for pant diapers to comprise an elastic material such as an elastic film or elastic strands at the waist level, taped diapers are more rarely fitted with a back elasticized waistband and even more rarely with an elasticized front waistband.

An elastic front waistband can provide additional leakage protection in the front of the diaper, especially when baby is lying on its belly, as it reduces or hinders any gap at the front edge of the diaper. Also, the stretchable front waistband can adjust the fit for baby in different activity/body stage, such as different lying positions. Combining with the stretchable back waistband, the whole baby body can obtain a 360 degree stretchable protection.

It has now been found that when the elastic force of the front waistband is too high, caregivers need to stretch the front waistband several times in order to flatten it enough so that the back ear tapes can be attached to the frontal landing zone due to the many wrinkles caused by the high front waist force. Even after the diaper is applied on the body, a too high front waistband force can result in a curved U shape deformation of the front waist edge of the diaper. This U shape causes the diaper to appear sagging, is not good for fit, and more importantly, a portion of the back tape may contact the skin, which can be disagreeable to the skin of the wearer.

There is thus a need for absorbent articles that address the above problems, in particular that have good fluid management properties, are easy to apply and are comfortable for the wearer during use.

### SUMMARY

In one form, a taped absorbent article comprises a front half region with a front waist edge, a back half region with a back waist edge, a first longitudinal edge and a second longitudinal edge, a wearer-facing side and a garment-facing side. The taped absorbent article comprises a topsheet on its wearer-facing side, a backsheet on its garment-facing side, an absorbent core between the topsheet and the backsheet, a pair of back ears each having a fastening tab that can be releasably attached to a landing zone on the garment-facing side of the absorbent article in the front half region of the article.

In another form, the absorbent article further comprises a front elastic waistband adjacent the front waist edge, and a back elastic waistband adjacent the back waist edge of the article. The invention is based on the finding the front waistband force should be within a specific relatively low range instead of a too high force. Accordingly the front elastic waistband should have a Front Waistband Elastic Force in the range from about 0.03N to about 0.32N, as measured by Waist Band Elastic Force Measurement Method disclosed herein. The back elastic waistband should have a Back Waistband Elastic Force which is higher than the Front Waistband Elastic Force.

The front and back waistband may be made from any suitable material, typically either a laminate comprising elastic strands or a laminate comprising a central elastic film layer, as is known in the art. In the first alternative, a continuous method for making the absorbent articles of the invention can comprise steps of:
providing a continuous supply of a waistband material in a machine direction, the material having a first longitudinal half and a second longitudinal half notionally divided by a folding line;
providing a continuous supply of elastic strands, wherein the elastic strands are adapted to provide the desired elastic strength for the respective waistbands;
intermittently applying an adhesive on the elastic strands;
superposing the elastic strands in machine direction over the waistband material on the first half of the waistband material;
folding the second half of the waistband material along the folding line over the first half of the waistband material thus obtaining a continuous waistband laminate comprising the elastic strands sandwiched between the folded waistband material, the laminate having adhesively bonded regions separated by non-bonded regions;
cutting this waistband laminate in cross-machine direction along dash cut lines in the non-bonded regions wherein the dash cut lines cut the elastic strands and the folded waistband material partially;
aligning the waistband laminate with the transversal direction of the chassis of the article;
stretching the waistband laminate so that it breaks along the dash cut lines to form combined pads;
cutting each combined pad along a cutting line in order to create discrete front and back waistband laminates; and
attaching the front and back waistbands to the chassis of the absorbent article.

This and further features or aspect are disclosed in the following description and the accompanying claims.

### BRIEF DESCRIPTION OF THE DRAWINGS

While the specification concludes with claims particularly pointing out and distinctly claiming the present invention, it is believed that the same will be better understood from the following description read in conjunction with the accompanying drawings in which:

FIG. 1 shows a perspective view of an exemplary taped diaper in a closed configuration as it would be when worn by a wearer;

FIG. 2 shows the wearer-facing side of the diaper of FIG. 1 with the diaper flattened out;

FIG. 3 shows the garment-facing side of the diaper of FIG. 1 with the diaper flattened out;

FIGS. 4-6 show different cross-sections of the layers of FIG. 2 in exploded view with the attachments such as adhesive being not represented for simplicity.

FIG. 7a-FIG. 7d illustrate a process for making simultaneously a front waistband and a back waistband;

FIGS. 8a-8b illustrates a waistband comprising a plurality of elastic strings adhesively sandwiched within a folded nonwoven;

FIG. 9a-9d shows the set-up of the test stand of Waist Band Force Measurement Method;

FIG. 10a-10e shows the different steps of Waist Band Force Measurement Method.

### DETAILED DESCRIPTION

### Definitions

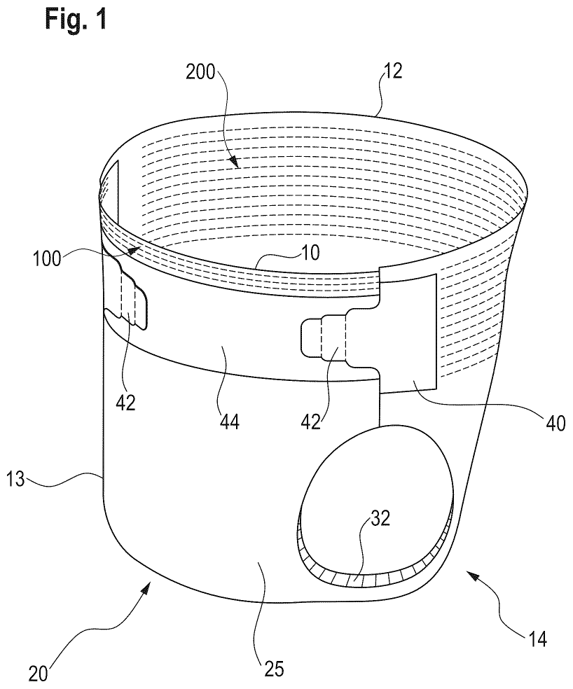

As used herein, the term “absorbent articles” or “diapers” refers to personal hygiene devices generally worn by babies, infants and incontinent adults about the lower torso so as to encircle the waist and legs of the wearer and that is specifically adapted to receive and contain urinary and fecal waste. These products are typically proposed as taped diapers or pant diapers. Taped diapers have a fastening system (as illustrated in FIG. 1), where the waist opening and leg openings are formed by releasably attaching the front waist region and back waist region to each other by a pair of back ears when the diaper is applied onto the wearer. In pant diapers, on the other hand, the articles have pre-formed waist opening and leg openings. A pant diaper is placed in position on the wearer by inserting the wearer's legs into the leg openings and sliding the pant diaper into position about the wearer's lower torso. A pant may be pre-formed by any suitable techniques including, but not limited to, joining together portions of the absorbent article using re-fastenable and/or non-refastenable bonds (e.g., seam, weld, adhesive, cohesive bond, fastener, etc.).

As used herein, the terms “nonwoven”, “nonwoven web” and “nonwoven layer” are used interchangeably. Nonwovens are broadly defined as engineered fibrous assemblies, primarily planar, which have been given a designed level of structural integrity by physical and/or chemical means, excluding weaving, knitting or paper making. The fibers may be of natural origin, such as cotton or bamboo fibers, or man-made origin. Synthetic fibers may be selected from the group consisting of polyolefins (such as polyethylene, polypropylene or combinations and mixtures thereof), polyethylene terephthalate (PET), co PET, polylactic acid (PLA), polyhydroxy alkanoid (PHA), or mixtures or combinations thereof. The fibers may be staple fibers (e.g., in carded nonwoven webs/layers) or continuous fibers (e.g., in spunbonded or meltblown nonwoven webs/layers).

Nonwoven materials can be formed by a variety of fiber materials (PP, PE, PET, coPET, bicomponent, and mixture thereof) and, in some cases, the fibers or the nonwovens can be treated to enhance specific fluid handling characteristics, such as fluid permeability or fluid barrier properties.

The term “dtex” as used herein refers to a unit used to indicate the fineness of a filament/fiber. The unit expresses the mass of a filament/fiber in grams per 10,000 meters of length.

“Longitudinal” refers to a direction running substantially perpendicular from a waist edge to an opposing waist edge of the article and generally parallel to the maximum linear dimension of the article (line 80 in FIG. 1). “Transversal” refers to a direction perpendicular to the longitudinal direction (line 90 in FIG. 1).

“Inner” and “outer” refer respectively to the relative location of an element or a surface of an element or group of elements. “Inner” implies the element or surface is oriented towards the inside of the article while “outer” implies the element or surface is oriented towards the outside of the article.

“Body-facing” and “garment-facing” refer respectively to the relative location of the surface of an element or group of elements. “Body-facing” implies the surface is nearer to the wearer during wear than the other surface of the element of group of elements. “Garment-facing” implies the surface is oriented away from the wearer during wear. The garment-facing surface may face another (i.e., other than the wearable article) garment of the wearer, other items, such as the bedding, or the atmosphere.

### General Description of an Exemplary Diaper

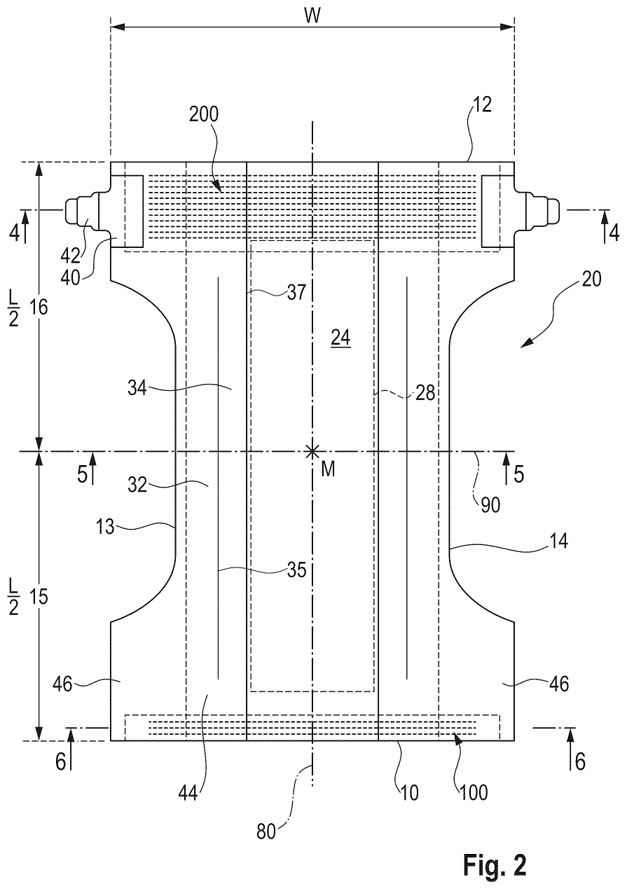

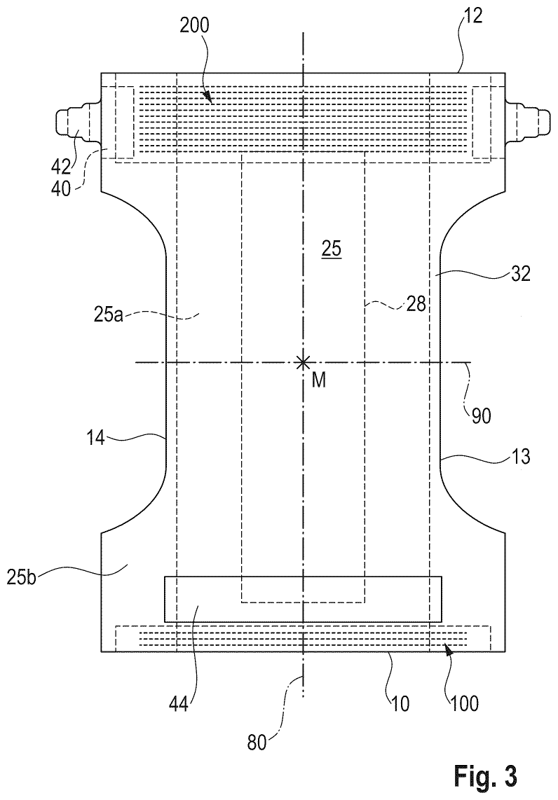

The absorbent articles of the invention can be generally described with reference to the taped diaper 20 shown in FIGS. 1-3. FIG. 1 is a perspective view of the exemplary diaper in a closed state as it would appear when worn by a wearer. The Figures are used herein as illustration and are not limiting the scope of the claims, unless specifically indicated to do so. In the following, the word “diaper” and “absorbent article” are used interchangeably. FIG. 2 shows a top view of the wearer-facing side of the diaper with the tapes open, and the diaper stretched flat, and FIG. 3 shows the same diaper from the garment-facing side.

The absorbent article has a front edge 10, a back edge 12, and two longitudinally-extending side (lateral) edges 13, 14. The front edge 10 is the edge of the article which is placed towards the front of the user when worn, and the back edge 12 is the opposite edge, and together form the waist opening of the diaper. The lateral edges 13, 14 respectively form the two leg openings.

The longitudinal edges 13, 14 of the diaper typically run generally parallel to the longitudinal centerline 80 of the diaper 20 and the front waist edge 10 and the back waist edge 12 typically run generally parallel to the transversal centerline 90 of the diaper 20. The front and back edges may be straight, as shown but they may also be wavy for example for a navel cut at the front.

As will be discussed in more details below, the exemplary topsheet represented in the Figures comprises a primary topsheet layer 24a, which is disposed centrally, and a wider secondary topsheet layer 24b which is disposed underneath the primary topsheet layer 24a. The secondary topsheet layer 24b may extend laterally up to the outer edges of 13, 14 of the diaper or stop laterally to an intermediate position with a barrier cuff material 36 extending on the wearer-facing side of the diaper. The primary topsheet layer and the secondary topsheet layer can be typically bonded together with an adhesive, the primary topsheet layer being mechanically deformed, so that the primary topsheet layer has a three-dimensional quilted soft feel. The exemplary taped diaper shown has a unibody construction, with shaped longitudinal edges 13, 14 formed by the combination of a leg cuff material 36 extending from the second topsheet layer 24b on the wearer-facing side and the backsheet outer cover 25b on the garment-facing side.

However, the disclosure is of course applicable to any kind of taped diaper constructions, in particular taped diapers having a chassis formed by a backsheet and topsheet having generally straight longitudinal edges, the diaper comprising discrete front ears and back ears that are attached to the chassis, as is also known in the art.

Before discussing the front waistband 100 and back waistband 200, the other components of the article are first generally discussed herein.

### Topsheet 24

The topsheet 24 is the part of the absorbent article 20 that is in contact with the wearer's skin. The topsheet 24 may be joined to portions of the backsheet 26, the absorbent core 28, the barrier leg cuffs 32-34, and/or any other layers as is known to those of ordinary skill in the art. Suitable topsheet are compliant, soft-feeling and non-irritating to the wearer's skin. The topsheet typically consists of or comprises at least a nonwoven layer. Further, topsheet is liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet is typically a single layer, however is may also have one or more layers, for example as represented in the Figure as a dual layer laminate construction comprising a three-dimensional centrally disposed primary topsheet layer 24a disposed on top of a larger secondary topsheet layer 24b.

The topsheet may be apertured, may have any suitable three-dimensional features, and/or may have a plurality of embossments (e.g., a bond pattern). Multi-component topsheet having three-dimensional materials are for example disclosed in WO 2017/156197. The topsheet may also be apertured by overbonding a material and then rupturing the overbonds through ring rolling. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, apertures will typically be present so that bodily exudates may pass through the topsheet.

### Backsheet Laminate 25

The backsheet 25 generally forms the garment-facing surface of the absorbent article. Typically, a backsheet is a laminate comprising a liquid-impermeable film 25a centrally disposed to at least cover the absorbent core 28 and which may extend beyond the absorbent core and an outer cover 25b. The backsheet film 25a is generally rectangularly shaped. The outer cover 25b is the outermost layer of the backsheet. When the absorbent article is formed by a unibody chassis as represented in the Figures, the outer cover 25b typically extend laterally beyond the longitudinal edges of the backsheet film 25a, so that the outer cover can form with the topsheet (or one layer thereof for a laminate) the external edges of the diaper, in particular the longitudinal edges 13, 14 of the diaper.

The backsheet film 25a may be joined to portions of the topsheet 24, the outer cover material 25b, the absorbent core 28, and/or any other layers of the absorbent article by any attachment methods known to those of skill in the art. The backsheet 25 prevents, or at least inhibits, the bodily exudates absorbed and contained in the absorbent core 28 from soiling articles such as bedsheets, undergarments, and/or clothing. The backsheet film is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

The outer cover (also referred to as a backsheet nonwoven) 25b may comprise one or more nonwoven materials joined to the backsheet film 25a. The outer cover material 25b forms at least a portion of the garment-facing surface of the absorbent article 20 and effectively “covers” the backsheet film 25a so that the film is not present on the garment-facing surface. The outer cover material 25b may comprise a bond pattern, apertures, and/or three-dimensional features. The outer cover material 25b may be a hydroentangled nonwoven material.

### Absorbent Core 28

As used herein, the term “absorbent core” refers to the component that absorbs and contains liquid such as urine received by the absorbent article. The absorbent core thus typically has a high absorbent capacity relative to the other components of the article. The absorbent core comprises an absorbent material and a core wrap (not represented separately for simplicity). As used herein, “absorbent core” does not include any acquisition-distribution systems, topsheet, or backsheet of the absorbent article.

The absorbent material typically comprises superabsorbent polymer particles as is known in the art. “Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test. The CRC measures the liquid absorbed by the superabsorbent polymer particles for free swelling in excess saline solution. The CRC is measured according to EDANA method NWSP 241.0.R2(19). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

The superabsorbent particles may be mixed with cellulose fibers, or may be immobilized within the core wrap using glue or a high loft nonwoven layer, as is known in the art. The core wrap typically comprises a top layer and a bottom layer, with the absorbent material being sandwiched between these layers. The core wrap may be a single material that is folded and attached to itself, or it may comprise a separate top layer and bottom layer that may be bonded or otherwise joined together.

A wide variety of absorbent cores may also be used. The absorbent material may form a rectangular layer but may also have a non-rectangular perimeter (“shaped” core), in particular, the absorbent material may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “sand-hour” for the area of the absorbent material.

The absorbent material may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam.

Various absorbent core designs comprising high amounts of SAP have been proposed in the past, see for example in U.S. Pat. No. 5,599,335 (Goldman), EP 1,447,066 (Busam), WO 95/11652 (Tanzer), US 2008/0312622 A1 (Hundorf), WO 2012/052172 (Van Malderen). In particular, the SAP printing technology as disclosed in US 2006/024433 (Blessing), US 2008/0312617 and US 2010/51166 A1 (both to Hundorf et al.) may be used. The absorbent core may also be an airfelt core comprising a high loft central nonwoven comprising interfibrous pores in which the superabsorbent polymer particles are at least partially distributed.

The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure. Such high loft cores are for example disclosed in CN 101797201, WO 2016106021 and U.S. patent application Ser. No. 17/542,592. The high loft central layer may typically be a nonwoven having in particular a density of less than 0.200 g/cc, measured at a pressure of 4. 14 kPa; with superabsorbent polymer particles (60) blended with the fibers of the central layer. The central layer may be for example a high loft carded nonwoven layer.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core.

The absorbent material layer may include one or more channel-forming area(s). Channel-forming areas are substantially free of absorbent material, in particular areas completely free of absorbent material (accidental minute amount of absorbent material due to involuntary contamination of the channels due to the high speed of the making process being disregarded). The channel-forming area(s) when present may comprise a channel bond between the top side of the core wrap and the bottom side of the core wrap. This bond provides for structural integrity of the channels in dry and wet state. Any known bonding techniques known in the art may be used to provide for this bond, but in particular a hotmelt adhesive bond may be used for the channel bond(s). An adhesive may be for example applied in the areas of the channels on the inner side of the top side and/or the inner side of the bottom side of the core wrap, typically by slot glue application or any other means, followed by the application of pressure in the areas of the channels to provide a good adhesive bonding in these areas. Exemplary patent disclosures of such adhesive bonding processes can be found for an airfelt or airfelt-free absorbent cores in WO 2012/170,798 A1 (Jackels et al.), EP 2,905,000 (Jackels et al.) and EP 2,905,001 (Armstrong-Ostle et al.).

### Inner Cuffs 34 and Outer Cuffs 32

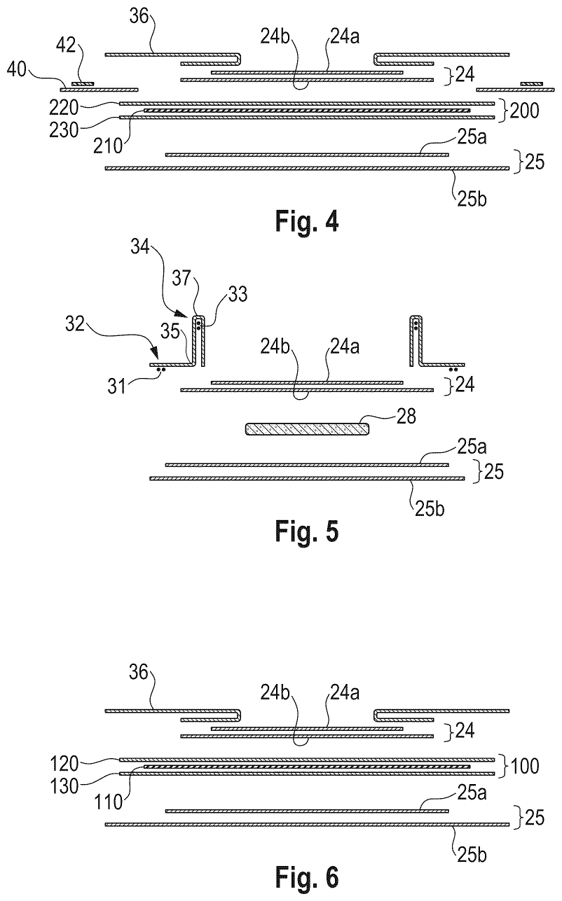

The absorbent article 20 may comprise a pair of barrier leg cuffs 34 (also called inner cuffs) and a pair of gasketing elastic cuffs 32 (also called outer cuffs), as is known in the art and as briefly explained herein. The barrier leg cuffs 34 are positioned laterally inboard of the gasketing cuffs 32 and can extend vertically away from the wearer-facing side of the article, as shown in FIG. 5. The barrier leg cuffs 34 are thus delimited by a proximal edge 35 joined directly or indirectly to the topsheet and/or the backsheet and a free terminal edge 37, which is intended to contact and form a seal with the wearer's skin. The barrier leg cuffs 34 typically extend partially between the front edge 10 and the back edge 12, disposed as a pair on opposite sides of the central longitudinal axis 80 and are at least present at the level of the M point. The barrier leg cuffs 34 may comprise one or more elastics strands 33 near or at the free terminal edge 37. These elastic strands 33 cause the barrier leg cuffs 34 to form a seal around the legs and torso of a wearer. The barrier leg cuffs 34 are not present towards the front edge and back edge of the article 20, rather the cuff material 36 is attached flat to the chassis of the article (typically on the topsheet) in the front and back region, as illustrated in FIG. 4 and FIG. 6 (the adhesive bonds are not represented for clarity of the Figures).

The gasketing elastic cuffs 32 are elasticized and also may comprise one or more elastic strands 31 sandwiched between the backsheet 25 and the barrier cuff material 36, and extend at least partially between the front end edge 10 and the back end edge 12 of the article, and are at least present at the level of the point M. The gasketing cuff's elastics 31 essentially cause portions of the absorbent article proximate to the chassis side edges 13, 14 to form a seal around the legs of the wearer.

Both inner cuffs 34 and outer cuffs 32 may be formed by a piece of cuff material 36, typically a nonwoven such as a SMS, which is partially bonded to the chassis of the absorbent article 20 from the longitudinal edges 13,14 up to the proximate edge 35 of the cuff, so that the barrier leg cuffs 34 can extend upwards from a wearer-facing surface of the absorbent article 20 and provide improved containment of body exudates approximately at the junction of the torso and legs of the wearer.

### Landing Zone 44

Referring to FIG. 1 and FIG. 3, the absorbent article 20 in the form of a taped diaper may have a discrete material forming a landing zone 44 on its garment-facing side, typically disposed proximate the front edge 10 of the article 20. The landing zone 44 is configured to receive the fasteners 42 and may comprise, for example, a plurality of loops configured to be engaged with, a plurality of hooks on the fasteners, or vice versa.

The landing zone 44 typically comprises a nonwoven material that is attached to a portion of the backsheet outer cover 25b adjacent to the front waist edge 10 of the taped article. Some outer cover backsheet material 25b may alternatively also serve directly as a landing zone without the need of a discrete landing zone strip 44, if the outer cover material has sufficient loft or other required properties to sufficient interact with the fasteners 42. When the landing zone 44 is present as a discrete piece of material attached to the backsheet, as shown in the Figures, the landing zone 44 is advantageously disposed underneath the front waistband 100. In other words, the front waistband 100 is advantageously disposed between the landing zone 44 and the front edge 10 of the article. The waistband 100 may be directly adjacent (i.e., less than 1 cm away from the front edge 10 of the article, and the landing zone 44 disposed still adjacent to the front edge (i.e. less than 5 cm away) but with the front waistband disposed in-between.

### Front Waistband 100 and Back Waistband 200

The front and back waistbands may be made of any elastic material suitable to incorporated into an absorbent article such as diaper as is known in the art. However, the elastic strength of the waistbands may chosen to match desired requirements.

A typical waistband construction 100, 200 comprise a plurality of elastic strands 110, 210 adhesively laminated between two nonwoven substrates. The elastic force of the waistbands can be adjusted by varying the elastic strength of the individual strands (typically thicker strand, as measured by their dtex value, have higher strengths) and the number of strands (the more numerous the strands are, the higher the elastic force achieved).

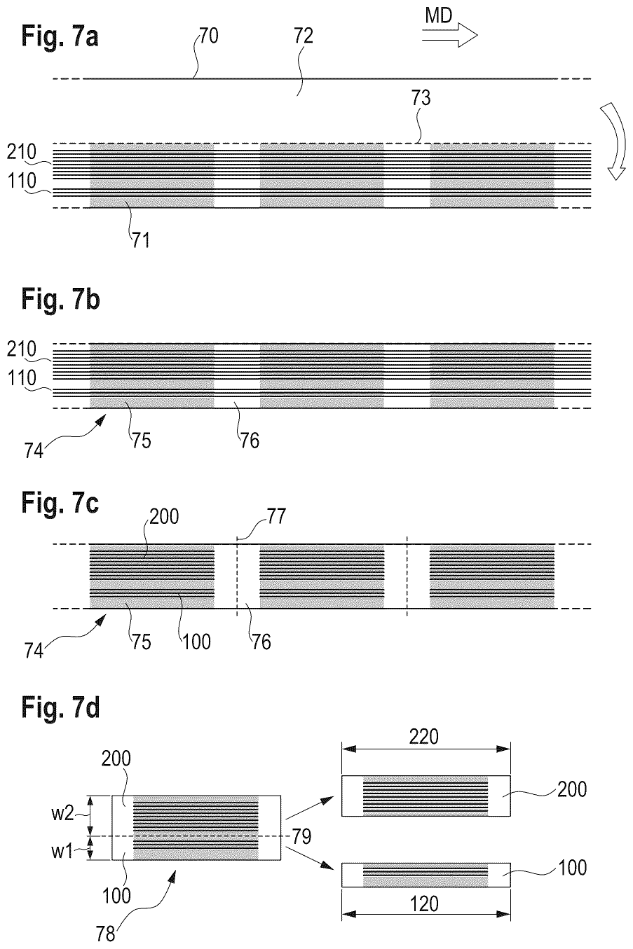

A simplified process to continuously and simultaneously make the front and back waistbands as a nonwoven laminate with elastic strands is illustrated in FIGS. 7a-d. A continuous supply of a waistband material 70 is provided in a machine direction MD, for example by unrolling a web of suitable material. A suitable waistband material is for example a low basis weight nonwoven (e.g., having a basis weight of from 8 gsm to 20 gsm) such as a SMS material. The web may advantageously have about twice the width of the combined front and back waistband desired, and has a first half 71 and second half 72 extending in the MD, which are of equal width and are notionally divided by a folding line 73.

The elastic strands 110, 210 are continuously provided from a suitable numbers of spools (not shown) as is known in the art. The elastic strands 110 and 210 may be the same elastomeric material, but advantageously the strands 110 for the front waistband 100 have a smaller dtex value and are less numerous than the strands 210 for the back waistband 200. The strands are intermittently applied with an adhesive typically through nozzles (not shown). Part of the elastic strands thus have no glue applied. The elastic strands 110, 210 are superposed with the waistband material 70 on the first half 71 of the waistband material. The skilled person understands that the process is continuous, with the waistband material 70 and the elastic strands 110, 210 being unrolled from rolls and/or spools of raw material and aligned in a parallel manner as shown in FIG. 7a in a machine direction at a high speed.

In a second step illustrated in FIG. 7b, the second half 72 of the waistband material 70 is folded by a folding plate (not shown) along the folding line 73 over the first half 71. This second step thus provides a laminate comprising the elastic strands sandwiched between two nonwoven layers formed by the folded nonwoven material web 70. The laminate may be passed between two rolls to help the adhesive providing integrity to the laminate. The continuous waistband laminate 74 thus obtained comprises the elastic strands 110, 210 sandwiched between the two nonwoven layers formed by the folded waistband material 70. The continuous waistband laminate thus display adhesively bonded regions 75 separated by non-bonded regions 76. The elastic strands are adhesively attached to the waistband material in the bonded regions 75.

The continuous laminate 74 is cut in cross-machine direction along dash cut lines 77 in the non-bonded regions 76 (FIG. 7c). These dash cut lines cut the elastic strands but only partially the waistband material (typically a nonwoven). The elastic strands snap back from the intermittent areas 76 where no glue was applied, while the waistband material 70 remains partially connected due to the dash cut process.

In a further step (not shown), the waistband laminate 74 is aligned with the transversal direction of the chassis of the article, optionally rotating the laminate construction through a 90 degrees rotating roll if needed. The laminate is also stretched so that it breaks along the dash cut lines 77 to form a combined pad 78. This combined pad 78 can then be cut along a cutting line 79 in another step (FIG. 7d) in order to divide the pad into a discrete front waistband laminate 100 and a back waistband laminates 200 that can be attached to the chassis of the article. This illustrated process is run simultaneously on the converting line at the same time as the rest of the chassis of the article is made.

However, other elasticized waistband making process and even other types of elastic waistbands may be used. For example, an alternative but more costly way to elasticize the waistband would be used an elastomeric film laminated between two nonwoven substrates, which could be made outside the line and then transported as a roll of material, as is known in the art. it is to be appreciated that the process described above is not limiting for articles which are defined by the properties of the waistbands.

It was found that the Front Waistband Elastic Force should be in the range from about 0.03 N to about 0.32 N. The back elastic waistband should have a Back Waistband Elastic Force which is higher than the Front Waistband Elastic Force. The Back Waistband Elastic Force may in particular be in the range of 1.1 N to about 2.9 N. The Front and Back Waistband Elastic Force are measured as indicated in the Measurement Method described further below.

The ratio of the value of the Back Waistband Elastic Force to the value of the Front Waistband Elastic Force value (Back Waistband Elastic Force divided by Front Waistband Elastic Force) may advantageously be in the range of 4 to 40, in particular in the range of 5 to 30.

Different Elastic Force for the Front Waistband and the Back Waistband can be obtained from the same type of elastomeric material (e.g., elastane strands) by providing more numerous elastic strands and/or strands having a higher dtex value in the back waistband than the elastic strands in the front waistband.

The stretched length of the front waistband 120 and the stretched length of the back waistband 220 may typically be about the same (typically corresponding to the length of the nonwoven material forming the outer layers of the elastic laminate). This is especially when the waistbands are made simultaneously from the same starting waistband material 70 as illustrated in FIGS. 7a-7d. The width w2 of the back waistband may typically be larger than the width w1 of the front waistband, as it comprises more elastic strands, and also there may be more space adjacent the back edge 12 of the article than adjacent to the front edge 10 of the article due to the presence of a landing in the front of the article.

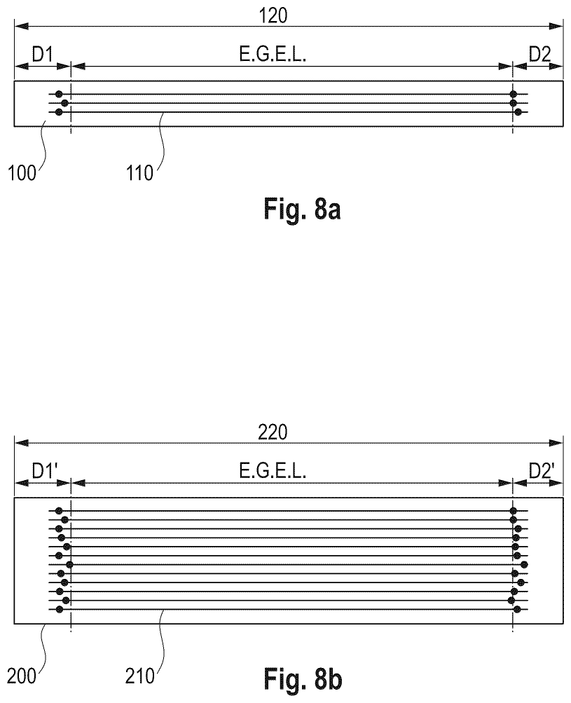

The Effective Glued Elastic Length (E.G.E.L. in FIG. 8) of each of the front waistband and the back waistband (measured as indicated in the Waist Band Force Measurement Method below) should be sufficiently long relative to the chassis of the diaper to provide a continuously extending elastic force around the waist of the wearer. The back waistband may advantageously have an Effective Glued Elastic Length in the range of from 50% to 100% (in particular from 70% to 95%) of the length W of the back edge 12 of the absorbent article (see FIG. 2), and likewise the front waistband may advantageously have an Effective Glued Elastic Length in the range of from 50% to 100% (in particular from 70% to 95%) of the length W of the front edge 10.

### Other Components

The topsheet 24, the backsheet 25, the absorbent core 28, the front and back waistbands 100, 200 and the other article's components may be assembled in a variety of well-known configurations, in particular by gluing, fusion and/or pressure bonding. The absorbent articles may comprise any typical layers and components used in absorbent products of the diaper type, and which are not necessarily represented in the simplified FIGS. 1-6. More detailed disclosures of example of such components are for example disclosed in WO 201493323, WO 2015/183669 (both Bianchi et al.), WO 2015/031225 (Roe et al.) or WO 2016/133712 (Ehrnsperger et al.) to name a few.

For example, the absorbent article may comprise at least one acquisition layer between the topsheet 24 and the absorbent core 28, as is common in the art. An acquisition layer is not necessary when the article comprise a dual layer topsheet laminate, as is represented for the absorbent article of the Figures as the secondary layer has a similar function. Acquisition layers are typically nonwovens, such as through-air bonded carded nonwovens, which may be hydrophillically treated. A distribution layer may also be present, in particular when it comprises or consists of relatively loose fibers with no or weak intra-fiber bonds and is disposed between the acquisition layer and the absorbent core. A typical example of such distribution material comprises or consists of cross-linked cellulose fibers. Examples of chemically cross-linked cellulosic fibers that have been used to make distribution layer are disclosed in U.S. Pat. Nos. 5,549,791, 5,137,537, WO 95/34329 or US 2007/118087. This type of material has been used in the past in disposable diapers as part of an acquisition-distribution system, for example in US 2008/0312622 A1 (Hundorf).

The topsheet 24, the backsheet 25, and the absorbent core 28 may be assembled in a variety of well-known configurations, in particular by gluing, heat embossing, ultrasonic bonding or combinations thereof. Exemplary diaper configurations are described generally in U.S. Pat. Nos. 3,860,003; 5,221,274; 5,554,145; 5,569,234; 5,580,411; and 6,004,306.

### Bio-Sourced Materials

Any components of the absorbent article can at least partially be comprised of bio-sourced content as described in US 2007/0219521 A1, US 2011/0139658 A1, US 2011/0139657 A1, US 2011/0152812 A1, US 2011/0139662 A1, and US 2011/0139659 A1. These components include, but are not limited to, topsheet nonwovens, backsheet films, backsheet nonwovens, barrier leg cuff nonwovens, superabsorbent material, upper and lower core wrap layer, adhesives, fastener hooks, and fastener landing zone nonwovens and film based.

The disposable absorbent article component may comprise a bio-based content value from about 10% to about 100% using ASTM D6866-10, method B, in another embodiment, from about 25% to about 75%, and in yet another embodiment, from about 50% to about 60% using ASTM D6866-10, method B.

In order to apply the methodology of ASTM D6866-10 to determine the bio-based content of any disposable absorbent article component, a representative sample of the disposable absorbent article component must be obtained for testing. Thereto, the disposable absorbent article component may be ground into particulates less than about 20 mesh using known grinding methods (e.g., Wiley® mill), and a representative sample of suitable mass taken from the randomly mixed particles.

### Example

An absorbent article suitable as a Size 4 baby diaper as generally illustrated in the FIGS. 1-6 was made with the following dimensions.

| | Dimensions in mm |
|---|---|
| Diaper length (L) | 480 |
| Core length | 375 |
| Diaper Pad width (W) | 335 |
| Front and Back Waistband Nonwoven Length | 310 |
| Glued waistband elastic width (E.G.E.L., 220) | 270 |
| Landing Zone (44) width | 230 |
| Backsheet Film (25a) width | 210 |
| 1st topsheet layer (24a) width | 110 |
| 2nd topsheet layer (24b) width | 142 |

The back waistband 200 comprised 11 elastane elastic strands each having a dtex of 620 and the front waistband 100 comprised 3 elastane elastic strands each having a dtex of 470. The front waistband had an elastic force (as measuredly the method indicated below) of 0.0N, and the back waistband an elastic force of 1.98N. The absorbent core was a high loft absorbent core as described above.

### Comparative Data

Four taped diapers having different front and back waistband elastic forces were tested in a consumer fit study (N=28 or 12 babies). A further control diaper was tested having only a back waistband but no front waistband. For each diapers, the accompanying parent was asked to grade the fit of the front waist of the diaper as Tight, Just Right or Loose.

The results are as indicated in Table 2 below:

TABLE 2
Fit Study Results

| Diaper tested | | #1 | #2 | #3 | #4 | #5 |
|---|---|---|---|---|---|---|
| Base Size | | 28 | 12 | 12 | 12 | 12 |
| Fit of Front | Tight | 21.4% | 8.3% | 8.3% | 0.0% | 0.0% |
| Waistband | Just right | 64.3% | 83.3% | 91.7% | 100% | 91.7% |
| | Loose | 14.3% | 8.3% | 0.0% | 0.0% | 8.3% |

| | | | | | |
|---|---|---|---|---|---|
| Front Waistband | 0.53N | 0.40N | 0.20N | 0.10N | N.A. |
| Elastic Force | | | | | |
| Back Waistband | 1.35N | 1.36N | 1.37N | 1.44N | 1.98N |
| Elastic Force | | | | | |
| Front/Back Elastic | 2.55 | 3.40 | 6.84 | 14.4 | N.A. |
| Force Ratio | | | | | |

Out of all the diapers tested, the diaper with the Front Elastic Waistband Force 0.10N was found to have the highest consumer fit rating (100% Just Right), as shown in the table 2 above.

While not wishing to be bound by theory, the inventors believe that a higher front waistband elastic force leads to higher loose fit perception due to the front waist edge of the diaper dropping into the anchoring position (much below the baby navel). This causes the diaper to sag and decreases the fit of the diaper. Furthermore, if the front waistband is too tight (high force), this causes many wrinkles in the front which cause consumers to stretch the front waist part several times. This increases the inconvenience of the application as adding additional application efforts. However, if the front waistband force is too low, there is no benefit in terms of fluid leakage prevention compared to a product without front elastic waistband.

### Test Methods

### Waist Band Elastic Force Measurement Method

This method measures the Waistband Elastic Force of the front and back elastic waistbands of the taped absorbent article at 85% of the Effective Glued Elastic Length (Lread) after two stretch at the Maximum stretch length (Lmax). The method is performed in a climate conditioned room at a standard condition of 23° C.±2° C. temperature.

### Definitions

Effective Glued Elastic Length (EGEL): for waistbands with one or multiple elastic strands, this is the minimum distance between the endings of the individual elastic strand at the position where they starts to pucker, as illustrated in FIGS. 8a-b by the distance E.G.E.L. for exemplary back waistbands 100, 200 (the same principle applies for a front waistband). For elastic waistband without elastic strands such as stretch laminates comprising an elastic film, the Effective Glued Elastic Length is measured at the position of the 1^{st} wrinkle on each side of the waistband. In practice the E.G.E.L. can be most easily measured by taking the known full length 120, 220 of the waistband material (in a relaxed state) and subtracting the length of the lateral non-bonded zones D1, D2 which can be directly measured on the waistbands.
Maximum stretch length Lmax: 95% of the Effective Glued Elastic Length of the waistband (Lmax=0.95×EGEL).
Reading point length Lread: 85% of the Effective Glued Elastic Length (Lread=0.85*EGEL).

### Sample Preparation:

1. Pick up a diaper product from package, open the product with topsheet facing to operator and do not stretch the waist band portion.
2. Construct a cut guiding line in parallel to the product transversal axis along the edge of waistband.
3. Cut the waistband along the line constructed using scissors/cutter, so that the waistband is separated from the chassis. Do not cut or tear into any elastic or nonwoven where elastics locate, otherwise pick up new pad and start over.
4. Tape the exposed edge closed using masking tape if any SAP exposure observed, ensuring that the tape does not constrain any material.
5. Draw a pair of reference lines (perpendicular to the length of the waistband) for each extremity of the Effective Glued Elastic Length as indicated in the definition above.

### Equipment:

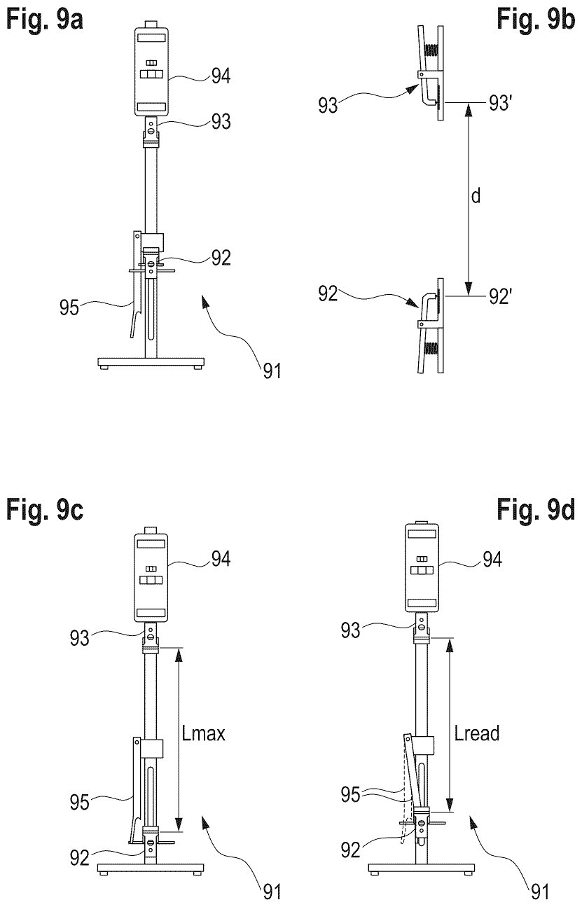

The measurement equipment 91 is illustrated in FIG. 9a-9b and comprises a lower clamp 92 and an upper clamp 93. The lower clamp 92 is slidable, so that the distance d between the upper and lower clamp bar lines 92′-93′ can be manually adjusted.

Force Stand: Force Gauge with Stand/Model E-DFE-002, Manufacturer: Trimborn+Guld Maschinenbau GmbH, with a digital force gauge 0-10 N (Newton) scale with a minimum resolution of 0.01 N, or equivalent (ref. number 94).

Clamp: Convenient source. Clamp that is wide enough to hold the Waistband strips.

Scissors: Convenient source.

Marker: e.g., Ballpoint pen.

Ruler: Metal Ruler traceable to NIST, DIN, JIS, CMC or other comparable National Standard, graduated in mm, longer than the length to be measured.

Timer: Convenient source.

Mask: Convenient source, against AGM.

Tape: Convenient source.

### Instrument Preparation:

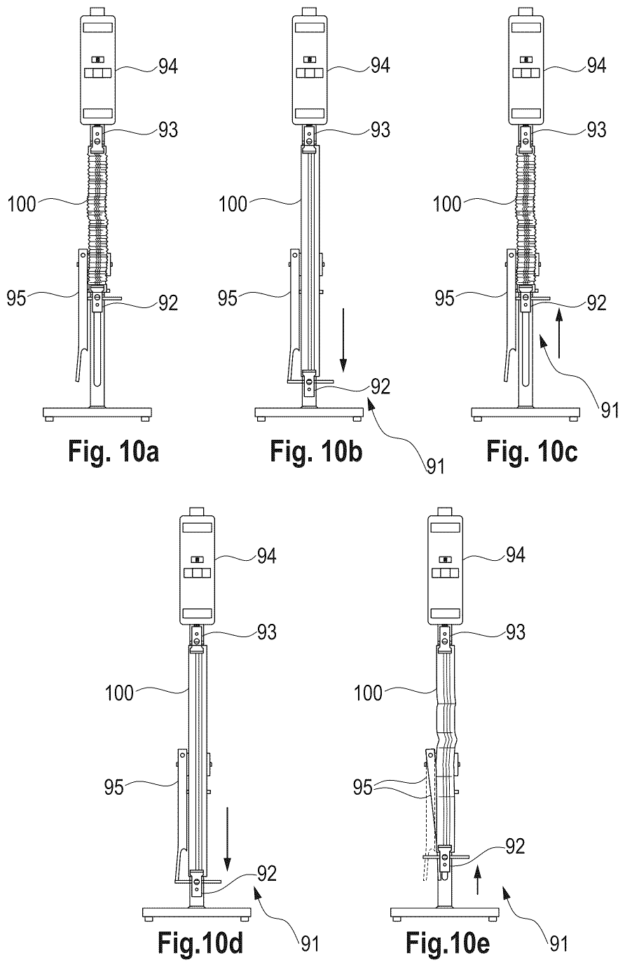

The Force Stand 91 is adjusted following below steps (FIGS. 9c-9d):
1. Move the lower clamp 92 to the lowest position.
2. Move and fix the lower clamp so that the distance d between the upper and lower wide clamp bar lines is Lₘₐₓ (FIG. 9c).
3. Move the lower clamp into the arm-stop 95 and adjust/fix the arm-stop so that the distance d between the upper clamp bar line 93′ and lower clamp bar line 92′ is L_{read} (FIG. 9d).
   Test Procedures (FIG. 10a-10e):
1. Position the lower clamp to its top position (in the relaxed position).
2. Mount the waistband strip vertically into the clamps by clipping one end of the waistband strip with all elastic ends into the upper clamp 93 so that the marked reference line (step 5 of the sample preparation) lines up exactly with the wide clamp bar line 93′. Make sure sample does not slip from clamping points.
3. Center the clamp directly over the sample.
4. Once the sample strip is mounted in the upper clamp, reset the force gauge.
5. Clip the other end of the sample strip with all elastic ends into the lower clamp 92 as described under step 2 so that the second marked reference line matches with the lower clamp line 92′. Make sure the sample strip is in its fully relaxed position (FIG. 10a). During this execution, do not stretch the sample strip. Make sure that the sample is not twisted and does not slip from clamping points.
6. As first step, the sample strip is extended: this is done by moving the lower clamp 92 to the bottom position (LMax). By doing this, the sample strip is extended to the “Maximum Extension” point (FIG. 10b). It is left there for approximately 1 second and then the sample strip is moved back to its fully relaxed position (FIG. 10c).
7. Extend the sample strip a second time to the LMax distance (FIG. 10d) and hold here for approximately 1 second.
8. Swing/slide in the stop arm 95 (which had set to be “reading point” at “Instrument Preparation” stage), and bring slowly (about 3-5 cm/s) the lower clamp 92 up against this “reading point” plate-stop arm (FIG. 10e).
9. Wait for approximately 5 seconds until the value on the gauge has stabilized, and read the value off the display. Record the value to the nearest 0.01N.

Note: If sample slip from clamp happened during measurement, redo the test for data reading. The lower clamp 92 is manually moved at a speed of about 10-15 cm/s in the steps above unless indicated otherwise.

### Reporting:

Report the determined values to the nearest 0.01 N.

### Misc

As used herein, the terms “comprise(s)” and “comprising” are open-ended; each specifies the presence of the feature that follows, e.g., a component, but does not preclude the presence of other features, e.g., elements, steps, components known in the art or disclosed herein. These terms based on the verb “comprise” should be read as encompassing the narrower terms “consisting essentially of” which excludes any element, step or ingredient not mentioned which materially affect the way the feature performs its function, and the term “consisting of” which excludes any element, step, or ingredient not specified. Any preferred or exemplary embodiments described below are not limiting the scope of the claims, unless specifically indicated to do so. The words “typically”, “normally”, “preferably”, “advantageously”, “in particular” and the likes also qualify features which are not intended to limit the scope of the claims unless specifically indicated to do so.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A taped absorbent article comprising:
a front half region with a front waist edge;
a back half region with a back waist edge;
a first longitudinal edge;
a second longitudinal edge;
a wearer-facing side;
a garment-facing side;
a topsheet on the wearer-facing side;
a backsheet on the garment-facing side;
an absorbent core between the topsheet and the backsheet;
a landing zone on the garment-facing side in the front half region;
a pair of back ears in the back half region, each back ear comprising a fastening tab that can be releasably attached to the landing zone;
a front elastic waistband adjacent the front waist edge, the front elastic waistband comprising a Front Waistband Elastic Force in a range from about 0.03 N to about 0.32 N, as measured by Waist Band Elastic Force Measurement Method disclosed herein; and
a back elastic waistband adjacent the back waist edge, the back elastic waistband comprising a Back Waistband Elastic Force which is higher than the Front Waistband Elastic Force.

2. The taped disposable absorbent article according to claim 1, wherein the Back Waistband Elastic Force is in a range of about 1.1 N to about 2.9 N.

3. The taped disposable absorbent article according to claim 1, wherein a ratio of the Back Waistband Elastic Force to the Front Waistband Elastic Force is in a range of about 4 to about 40.

4. The taped disposable absorbent article according to claim 1, wherein a ratio of the Back Waistband Elastic Force to the Front Waistband Elastic Force is in a range of about 5 to about 30.

5. The taped disposable absorbent article according to claim 1, wherein the front elastic waistband and the back elastic waistband each comprise a plurality of elastic strands.

6. The taped disposable absorbent article according to claim 5, wherein the elastic strands of the back waistband are more numerous and/or comprise a higher dtex value than the elastic strands of the front waistband.

7. The taped disposable absorbent article according to claim 1, wherein the elastic strands of each of the front elastic waistband and the back elastic waistband are sandwiched by a nonwoven waistband material.

8. The taped disposable absorbent article according to claim 7, wherein the nonwoven waistband material is a SMS material.

9. The taped disposable absorbent article according to claim 1, wherein the front elastic waistband is disposed between the landing zone and the front waist edge.

10. The taped disposable absorbent article according to claim 1, wherein the back elastic waistband comprises an Effective Glued Elastic Length in a range of from about 50% to about 100% of a length of the back waist edge.

11. The taped disposable absorbent article according to claim 10, wherein the back elastic waistband comprises an Effective Glued Elastic Length in a range of from about 70% to about 95% of the length of the back waist edge.

12. The taped disposable absorbent article according to claim 1, wherein the front elastic waistband comprises an Effective Glued Elastic Length in a range of from about 50% to about 100% of a length of the front waist edge.

13. The taped disposable absorbent article according to claim 12, wherein the front elastic waistband comprises an Effective Glued Elastic Length in a range of from about 70% to about 95% of the length of the front waist edge.

14. The taped disposable absorbent article according to claim 1, wherein the absorbent core comprises a high loft nonwoven central layer in a core wrap, wherein superabsorbent polymer particles are at least partially immobilized within pores of the high loft nonwoven central layer.

15. The taped disposable absorbent article according to claim 1, comprising a dual layer topsheet comprising a primary three-dimensional topsheet layer laminated to a larger secondary nonwoven topsheet layer.

16. The taped disposable absorbent article according to claim 1, comprising renewable or recyclable material.

17. A package comprising a plurality of absorbent articles according to claim 1.

18. A method for continuously making a plurality of absorbent articles according to claim 1, comprising steps of:
providing a continuous supply of a waistband material in a machine direction, the waistband material comprising a first half and a longitudinal half extending in the machine direction notionally divided by a folding line;
providing a continuous supply of elastic strands, wherein the elastic strands are adapted to provide an elastic strength for the front elastic waistband and the back elastic waistband;
intermittently applying an adhesive on the elastic strands;
superposing the elastic strands in the machine direction over the waistband material on the first half of the waistband material;
folding a second half of the waistband material along the folding line over the first half of the waistband material thus obtaining a continuous waistband laminate comprising the elastic strands sandwiched between the folded waistband material, the laminate comprising adhesively bonded regions separated by non-bonded regions;
cutting the waistband laminate in a cross-machine direction along dash cut lines in the non-bonded regions wherein the dash cut lines cut the elastic strands and the folded waistband material partially;
aligning the waistband laminate with a transversal direction of the article;
stretching the waistband laminate so that the waistband laminate breaks along the dash cut lines to form combined pads;
cutting each combined pad along a cutting line in order to create discrete front and back waistband laminates;
attaching the front and back waistband laminates to the absorbent article.
